SEQUENCE LISTING (6 sequences)

SEQ ID NO: 1 - DNA, 1284 bp, Escherichia coli
atggaatccctgacgttacaacccatcgctcgtgtcgatggcactattaatctgcccggttccaagagcgtttctaaccgcgctttattgctggcggcattagcacacggcaaaacagtattaaccaatctgctggatagcgatgacgtgcgccatatgctgaatgcattaacagcgttaggggtaagctatacgctttcagccgatcgtacgcgttgcgaaattatcggtaacggcggtccattacacgcagaaggtgccctggagttgttcctcggtaacgccggaacggcaatgcgtccgctggcggcagctctttgtctgggtagcaatgatattgtgctgaccggtgagccgcgtatgaaagaacgcccgattggtcatctggtggatgcgctgcgcctgggcggggcgaagatcacttacctggaacaagaaaattatccgccgttgcgtttacagggcggctttactggcggcaacgttgacgttgatggctccgtttccagccaattcctcaccgcactgttaatgactgcgcctcttgcgccggaagatacggtgattcgtattaaaggcgatctggtttctaaaccttatatcgacatcacactcaatctgatgaagacgtttggtgttgaaattgaaaatcagcactatcaacaatttgtcgtaaaaggcgggcagtcttatcagtctccgggtacttatttggtcgaaggcgatgcatcttcggcttcttactttctggcagcagcagcaatcaaaggcggcactgtaaaagtgaccggtattggacgtaacagtatgcagggtgatattcgctttgctgatgtgctggaaaaaatgggcgcgaccatttgctggggcgatgattatatttcctgcacgcgtggtgaactgaacgctattgatatggatatgaaccatattcctgatgcggcgatgaccattgccacggcggcgttatttgcaaaaggcaccaccacgctgcgcaatatctataactggcgtgttaaagagaccgatcgcctgtttgcgatggcaacagaactgcgtaaagtcggcgcggaagtggaagaggggcacgattacattcgtatcactcctccggaaaaactgaactttgccgagatcgcgacatacaatgatcaccggatggcgatgtgtttctcgctggtggcgttgtcagatacaccagtgacgattcttgatcccaaatgcacggccaaaacatttccggattatttcgagcagctggcgcggattagccaggcagcctaa

SEQ ID NO: 2 - DNA, 240 bp, Neurospora crassa
atcggccccaacacctgctccatcgacgactacaagccttactgctgccagtctatgtccggccccgccggctcccctggtctcctcaacctcatccccgtcgacctcagcgcctcgctcggctgcgttgtcggtgtcatcggctcccaatgtggtgccagcgtcaagtgctgcaaggacgatgttaccaacaccggcaactccttcctcatcatcaacgctgccaactgcgttgcctaa

SEQ ID NO: 3 - DNA, 1572 bp, Artificial Sequence
  fusions protein of EPSPS/Ccg2
atcggccccaacacctgctccatcgacgactacaagccttactgctgccagtctatgtccggccccgccggctcccctggtctcctcaacctcatccccgtcgacctcagcgcctcgctcggctgcgttgtcggtgtcatcggctcccaatgtggtgccagcgtcaagtgctgcaaggacgatgttaccaacaccggcaactccttcctcatcatcaacgctgccaactgcgttgccgaattcggcggcggcggcagcggcggcggcggcagcggcggcggcggcagcatggaatccctgacgttacaacccatcgctcgtgtcgatggcactattaatctgcccggttccaagagcgtttctaaccgcgctttattgctggcggcattagcacacggcaaaacagtattaaccaatctgctggatagcgatgacgtgcgccatatgctgaatgcattaacagcgttaggggtaagctatacgctttcagccgatcgtacgcgttgcgaaattatcggtaacggcggtccattacacgcagaaggtgccctggagttgttcctcggtaacgccggaacggcaatgcgtccgctggcggcagctctttgtctgggtagcaatgatattgtgctgaccggtgagccgcgtatgaaagaacgcccgattggtcatctggtggatgcgctgcgcctgggcggggcgaagatcacttacctggaacaagaaaattatccgccgttgcgtttacagggcggctttactggcggcaacgttgacgttgatggctccgtttccagccaattcctcaccgcactgttaatgactgcgcctcttgcgccggaagatacggtgattcgtattaaaggcgatctggtttctaaaccttatatcgacatcacactcaatctgatgaagacgtttggtgttgaaattgaaaatcagcactatcaacaatttgtcgtaaaaggcgggcagtcttatcagtctccgggtacttatttggtcgaaggcgatgcatcttcggcttcttactttctggcagcagcagcaatcaaaggcggcactgtaaaagtgaccggtattggacgtaacagtatgcagggtgatattcgctttgctgatgtgctggaaaaaatgggcgcgaccatttgctggggcgatgattatatttcctgcacgcgtggtgaactgaacgctattgatatggatatgaaccatattcctgatgcggcgatgaccattgccacggcggcgttatttgcaaaaggcaccaccacgctgcgcaatatctataactggcgtgttaaagagaccgatcgcctgtttgcgatggcaacagaactgcgtaaagtcggcgcggaagtggaagaggggcacgattacattcgtatcactcctccggaaaaactgaactttgccgagatcgcgacatacaatgatcaccggatggcgatgtgtttctcgctggtggcgttgtcagatacaccagtgacgattcttgatcccaaatgcacggccaaaacatttccggattatttcgagcagctggcgcggattagccaggcagcctaa

SEQ ID NO: 4 - protein, 427 aa, Escherichia coli
MESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 5 - protein, 79 aa, Artificial Sequence
  CCg2 of Neurospora crassa optimized
IGPNTCSIDDYKPYCCQSMSGPAGSPGLLNLIPVDLSASLGCVVGVIGSQCGASVKCCKDDVTNTGNSFLIINAANCVA

SEQ ID NO: 6 - protein, 523 aa, Artificial Sequence
  Fusionsprotein EPSPS/Ccg2
IGPNTCSIDDYKPYCCQSMSGPAGSPGLLNLIPVDLSASLGCVVGVIGSQCGASVKCCKDDVTNTGNSFLIINAANCVAEFGGGGSGGGGSGGGGSMESLTLQPIARVDGTINLPGSKSVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIDMDMNHIPDAAMTIATAALFAKGTTTLRNIYNWRVKETDRLFAMATELRKVGAEVEEGHDYIRITPPEKLNFAEIATYNDHRMAMCFSLVALSDTPVTILDPKCTAKTFPDYFEQLARISQAA

(19) United States
(12) Patent Application Publication
(10) Pub. No.: US 2022/0026421 A1
(43) Pub. Date: Jan. 27, 2022

(54) METHOD, SURFACE, PARTICLE AND KIT FOR THE DETECTION OF ANALYTES IN SAMPLES

(71) Applicants: TECHNISCHE UNIVERSITÄT DRESDEN, Dresden (DE); UNIVERSITÄT LEIPZIG, Leipzig (DE)

(72) Inventors: Gerhard RÖDEL, Dresden (DE); Kai OSTERMANN, Dresden (DE); Julia DÖRING, Dresden (DE); Christian DAHMANN, Dresden (DE); Tilo POMPE, Leipzig (DE); David RETTKE, Leipzig (DE); Steve MARTIN, Leipzig (DE)

(73) Assignees: TECHNISCHE UNIVERSITÄT DRESDEN, Dresden (DE); UNIVERSITÄT LEIPZIG, Leipzig (DE)

(21) Appl. No.: 17/297,150

(22) PCT Filed: Nov. 18, 2019
(86) PCT No.: PCT/EP2019/081614

§ 371 Date: May 26, 2021

(30) Foreign Application Priority Data
Nov. 28, 2018 (DE) 10 2018 130 134.0

(51) Int. Cl. G01N 33/53; G01N 33/543; G01N 33/545; C07K 14/37; C12N 9/10

(52) U.S. Cl. CPC G01N 33/5308; G01N 33/54313; G01N 33/545; G01N 2430/20; C12N 9/1092; C12Y 205/01019; C07K 2319/00; C07K 14/37

(57) ABSTRACT

The invention relates to a method, to a surface, to a particle, and to a kit for the detection of low molecular weight analytes such as crop protection agents in samples. In particular, the invention relates to a method for the detection of glyphosate through protein-functionalised surfaces and functionalised particles by means of reflection interference contrast microscopy (RICM).

The invention relates to a method, to a surface, to a particle and to a kit for the detection of low molecular weight analytes such as crop protection agents in samples. In particular, the invention relates to a method for the detection of glyphosate through protein-functionalised surfaces and functionalised particles by means of reflection interference contrast microscopy (RICM).

The detection of low molecular weight analytes is currently very complex. In particular, the detection of crop protection agents in drinking water, food and soil samples and the health effects thereof are currently of great interest. The main problem appears to be the contamination of food, including drinking water, with glyphosate.

The detection of contamination with crop protection agents in food is carried out according to the multi-method DFG S19, whereby various extraction steps are carried out for sample preparation and subsequently the laboratory analysis by means of LC-MS or GC-MS takes place.

A method for the detection of analytes is also known from EP2 752 664 A1. The immobilisation of an analyte on hydrogel particles takes place thereby, which particles subsequently interact with a ligand immobilised on a surface. Depending on the degree of interaction, the hydrogel particles are deformed by accumulation on the surface, so that the deformation takes place by means of reflection interference contrast microscopy. A detection or characterisation of the analyte can thereby take place.

US 2014/0255916 A1 discloses a method and a kit for measuring the ability of a test sample to inhibit the binding of a pathogen receptor, preferably a sialic acid receptor, to a host cell ligand of the pathogen. The method comprises an immobilised receptor which is contacted with a test sample and a particle reagent containing the ligand, preferably sialic acid, the particle reagent being a biological reagent selected from erythrocytes, erythrocyte vesicles, erythrocyte ghost cells, membrane fragments, membrane vesicles, proteins and combinations, in particular in the form of colloids, beads, or combinations thereof; wherein the particle can be coated and/or magnetic, electrically conductive and/or semiconducting. The amount of particle reagent bound to the surface is then measured.

Various methods for the determination of glyphosate are also known in the prior art.

CN 102207495 A discloses a method for the determination of the glyphosate content in soil samples by means of HPLC.

WO 00/14538 discloses a linker-assisted immunoassay for glyphosate and a method for the production of glyphosate antibodies comprising the production of glyphosate conjugates with a carrier molecule, preferably porcine thyroglobulin, bovine serum albumin, human serum albumin, ovalbumin or keyhole limpet haemocyanin; and immunising a host with the conjugate. The linker-assisted immunoassay for the detection of the analyte in a test sample comprises the production of a linker-analyte conjugate by means of a test sample, contacting with a glyphosate antibody and contacting the test sample with a solid phase with an immobilised second carrier molecule which is covalently coupled to glyphosate, to a glyphosate derivative or to a glyphosate salt. The second carrier molecule is selected from porcine thyroglobulin, bovine serum albumin, human serum albumin, ovalbumin or keyhole limpet haemocyanin, but differs from the first carrier molecule, which is used for the production of the glyphosate antibody. The amount of bound antibody, which is indirectly proportional to the amount of glyphosate in the test sample, is then determined on the solid phase. The detection is preferably carried out by means of enzymatic detection, the antibody being conjugated with biotin and a labelled enzyme being added which binds biotin. The enzyme is preferably alkaline phosphatase or horseradish peroxidase.

WO 2008118899 A1 also discloses a method for the detection of glyphosate by means of LC/GC coupled MS. Alternatively, detection methods by means of bioluminescence (WO2010104861A1) and ELISA (WO2000014538A1) are also described.

WO 2018057647 A1 describes an assay for a biochip in which pesticides can be detected on a sensor platform. The detection is based on the amplification of corresponding nucleic acids, which leads to significantly long analysis times in the range of a plurality of hours.

US 2005/0118665 A1 discloses a method for the determination of an enzymatic reaction, wherein at least one protein and at least one substance are immobilised on a solid support at a distance sufficient for an enzymatic reaction by covalent binding or by using a fusion protein, preferably with a His-tag and a nickel-coated surface; and are incubated under conditions for an enzymatic reaction. The substance is preferably a known substrate for an examined enzymatic reaction and the protein is examined for an enzymatic activity. The enzyme is preferably an oxidoreductase, a transferase, a hydrolase, a lyase, an isomerase or a ligase.

DE 10 2011 089 241 B3 describes a method for coating a substrate with a hydrophobin bilayer and a substrate with a hydrophobin bilayer coating. Furthermore, DE 10 2011 089 241 B3 discloses a substrate coated with a hydrophobic fusion protein. The hydrophobic fusion protein preferably comprises a functional domain, it being possible for the functional domain to be a protein domain with enzyme activity.

The object of the present invention is therefore to provide a method for the detection of an analyte which overcomes the disadvantages in the prior art.

The object is achieved by a method according to claim 1. Advantageous embodiments are specified in the dependent claims.

According to the invention, a method for the detection of analytes is proposed comprising the steps of:
providing a surface with an immobilised analyte binding partner,
contacting the analyte binding partner with a sample containing the analyte, wherein the analyte interacts with the analyte binding partner,
contacting the analyte binding partner with a competitor, wherein the competitor is immobilised on a particle and interacts with the analyte binding partner,
detecting the competitors bound to the analyte binding partner,
   wherein the analyte is glyphosate,
   wherein the analyte binding partner has the active centre of the enzyme 5-enolpyruvylshikimate-3-phosphate synthase (EPSPS) as the analyte binding site, and wherein the particle is designed to be deformable.

Low molecular weight analytes are understood to mean substances or molecules having a molar mass of <800 g/mol.

In embodiments of the invention, the contact of the analyte binding partner with the analyte takes place prior to the contact with the particle for a period of 1 min to 30 min, preferably 1 min to 20 min, particularly preferably 1 min to 10 min, most preferably 1 min to 7 min.

In an alternative embodiment, simultaneous contacting of the analyte binding partner with the analyte and the particle takes place.

In embodiments of the invention, the particle is a functionalised particle, the surface of the particle having a functionalisation. The functionalisation is used to bind the competitor. For example, the functionalisation can take place through chemical groups.

In embodiments of the invention, the particle is a hydrogel particle.

In embodiments of the invention, the particle has a diameter of 10 μm to 100 μm, particularly preferably a diameter of around 25 μm.

In embodiments of the invention, the particle has a modulus of elasticity in the range from 10 kPa to 100 kPa, particularly preferably in the range from 15 kPa to 50 kPa. In this way, a high level of sensitivity is advantageously ensured. At the same time, uneven deformation of the particles is excluded.

The embodiments in terms of diameter and modulus of elasticity (here Young's modulus of elasticity) of the particles determine, among other things, the sensitivity and reliability of the analysis method, whereby the diameter can be determined by optical brightfield microscopy and the modulus of elasticity can be determined from force-distance curves from atomic force spectroscopy.

In embodiments of the invention, the particle has a carboxy functionalisation. The synthesis and carboxy functionalisation of the particles, in particular the hydrogel microparticles, took place according to the method described by Pussak et al. (Pussak, et al., 2012) via emulsion and radical precipitation polymerisation of polyethylene glycol diacrylamide or polyethylene glycol diacrylate with subsequent radical grafting of acrylic acid monomers, crotonic acid monomers or other alkene derivatives with functional groups such as amines for the introduction of the carboxyl, amino or other functional groups.

In embodiments, the synthesis and carboxy functionalisation of the particles takes place microfluidically by means of photoinitiated radical crosslinking of polyethylene glycol diacrylamide or polyethylene glycol diacrylate (preferred molecular weight in the range from 500 Da to 8,000 Da, particularly preferably around 4,000 Da) with subsequent photoinitiated radical grafting of acrylic acid monomers to introduce the carboxyl groups, monodisperse particles having a diameter in the range from 10 μm to 100 μm, particularly preferably around 25 μm, being produced.

In embodiments of the invention, the competitor is immobilised on the particle via a linker. The linker is attached to the functionalised particle surface.

The respective linker molecule allows suitable immobilisation of the competitor via the carboxyl or secondary amino group, the coupling group influencing the functionality and sensitivity of the method. Furthermore, the resulting affinity of the immobilised competitor can be varied via the length or the degree of polymerisation of the linker and thus the working range of the method can be set.

In embodiments of the invention, the linker is selected from the groups of homo- and heterobifunctional linkers and comprises, for example, ethylenediamine, oligo- and polyethylene glycol bisamines, peptides such as pentaglycine and amino acids or a bifunctional linker having other groups such as thiols or azides.

In embodiments of the invention, the linker has a contour length (L) and a degree of polymerisation (PG). In embodiments of the invention, the linker has a contour length (L) in the range from 5 Å to 200 Å, preferably in the range from 10 Å to 50 Å.

In embodiments of the invention, the linker has a degree of polymerisation in the range from 1 to 70, preferably in the range from 3 to 20.

Examples of suitable linkers are ethylenediamine: L=5.3 Å; PG=1 or pentaglycine: L=16.3 Å; PG=5 or PEG-bisamine 3000: L=191 Å; PG=68.

In embodiments of the invention, the linkers contain protecting groups. In embodiments of the invention, the protecting group is selected from fluorenylmethoxycarbonyl, tert-butyloxycarbonyl or tert-butyl protecting groups. In this way, it is advantageously ensured that no undesired polymerisation of the linker molecules or cross-linking of the particles occurs.

According to the invention, the competitor is designed to interact with the analyte binding partner, the competitor and the analyte competing for the interaction with the analyte binding partner.

This creates a competition between the free analyte in a sample and the particle-bound competitor, an equilibrium being established as a function of the analyte concentration. The more free analyte is present, the lower the binding of the particle-bound competitors to the analyte binding partner. As a result, the contact area between the particle and the surface is reduced. In the opposite case, if little analyte is present in the sample to be examined, there is an increased binding of the particle-bound competitors to the analyte binding partner, the contact area between particle and surface increasing. In embodiments of the invention, the competitor is identical to the analyte.

In embodiments of the invention, the competitor is selected from phosphoenolpyruvate, phosametine (Huangcaoling), substrate analogues of the enzyme 5-enolpyruvylshikimate-3-phosphate synthase (EPSPS) or glyphosate.

Glyphosate (N-(phosphonomethyl)glycine) is a non-selective leaf herbicide (broad-spectrum or total herbicide) with systemic effects that is absorbed through all green parts of the plant. Glyphosate is used in agriculture against monocotyledonous and dicotyledonous weeds in agriculture, wine and fruit growing, in the cultivation of ornamental plants, on meadows, pastures and lawns and in the forest. The leaves take up glyphosate by diffusion and glyphosate is distributed in the plant via the phloem. Glyphosate works by blocking the enzyme 5-enolpyruvylshikimate-3-phosphate synthase (EPSPS), which is required for the synthesis of the aromatic amino acids phenylalanine, tryptophan and tyrosine via the shikimate pathway in plants, as well as in some microorganisms. Due to the similarity of glyphosate with the natural substrate phosphoenolpyruvate of the enzyme 5-enolpyruvylshikimate-3-phosphate synthase (EPSPS), this enzyme is inhibited.

The term “substrate analogues” is understood to mean compounds which bind to the analyte binding partner, in particular to the active centre of the enzyme 5-enolpyruvylshikimate-3-phosphate synthase (EPSPS). In embodiments, substrate analogues comprise substrates, in particular shikimate-3-phosphate (S3P) or phosphoenolpyruvate (PEP); functional substrate analogues, in particular phosametine; and competitive inhibitors, in particular glyphosate. Marzabadi et al. and Priestman et al. disclose further substrate analogues of the enzyme 5-enolpyruvylshikimate-3-phosphate synthase (EPSPS), in particular (3R,4S,5R)-5-amino-4-hydroxy-3-(phosphonooxy)cyclohex-1-en-1-carboxylic acid, 1-carboxy-1-(phosphonooxy)-ethan-1-ylium, (3R,4S,5R)-5-((S)-1-carboxy-1-phosphonoethoxy)-4-hydroxy-3-(phosphonooxy)-cyclohex-1-en-1-carboxylic acid, (3R,4S,5R)-5-((R)-1-carboxy-1-phosphonoethoxy)-4-hydroxy-3-(phosphonooxy)-cyclohex-1-en-1-carboxylic acid, (2R,3aR,7R,7aS)-2-methyl-7-(phosphonooxy)-3a,4,7,7a-tetrahydrobenzo[d][1,3]-dioxole-2,5-dicarboxylic acid, (3R,4S,5R)-5-((carboxymethyl)-(phosphonomethyl)amino)-4-hydroxy-3-(phosphonooxy)-cyclohex-1-en-1-carboxylic acid, (3R,4S,5R)-5-((carboxymethyl)-(phosphonomethyl)amino)-3,4-di-hydroxycyclohex-1-en-1-carboxylic acid [3,4].

In further embodiments of the invention, both competitor and analyte are glyphosate.

In embodiments of the invention, the analyte binding partner is designed as a fusion protein. The fusion protein has, for example, different protein domains which have different functionalities.

According to the invention, the analyte binding partner comprises a protein domain having an analyte binding site. The analyte binding site is used to bind the analyte to the analyte binding partner.

In embodiments of the invention, the analyte binding site is designed as a binding pocket of an enzyme or an allosteric binding site for the analyte.

According to the invention, the analyte binding partner has the active centre of the enzyme 5-enolpyruvylshikimate-3-phosphate synthase (EPSPS) as the analyte binding site. The distance between the binding pocket and the protein surface in the EPSPS is approx. 10 Å in the closed conformation.

In embodiments of the invention, the analyte binding partner comprises a protein domain selected from hydrophobins, ECM proteins, S-layer proteins, peptide linkers and protein tags. This allows directed immobilisation to the surface, at least one protein domain mediating the binding and a further protein domain having a further functionality. The analyte binding partner preferably has a hydrophobin domain. Hydrophobins advantageously immobilise themselves in a self-assembling manner on different material surfaces and geometries.

In embodiments of the invention, the analyte binding partner has a hydrophobin domain Ccg2 from Neurospora (N.) crassa. The analyte binding partner preferably has SEQ. ID. No. 5.

In embodiments of the invention, the analyte binding partner is a fusion protein having a hydrophobin domain and a 5-enolpyruvylshikimate-3-phosphate synthase domain. The hydrophobin domain allows for the immobilisation of the analyte binding partner on the surface. In embodiments of the invention, the fusion protein has SEQ ID No. 6.

In embodiments of the invention, the analyte binding partner is immobilised on the surface in a mixture with hydrophobins, the mixture of the analyte binding partner with the hydrophobin being in the range from 1:2 to 1:10, preferably between 1:3 to 1:8, particularly preferably around 1:5. For the purposes of the present invention, the specified mixing ratios relate to the molar mass. Advantageously, with mixing ratios in the range from 1:3 to 1:8, particularly homogeneous and not very rough surfaces are obtained, as a result of which the particle binding to the surfaces is improved.

In embodiments of the invention, a mixture of the fusion protein having a hydrophobin domain and a 5-enolpyruvylshikimate-3-phosphate synthase domain (SEQ ID No. 6) and the hydrophobin domain Ccg2 from Neurospora (N.) crassa (SEQ ID No. 5) takes place in a ratio of 1:2 to 1:10, preferably between 1:3 to 1:8, particularly preferably around 1:5, most preferably 1:5 for the immobilisation of the analyte binding partner. By varying the ratio of fusion protein and hydrophobin on the surface, the sensitivity to glyphosate can also be set in a very targeted manner and thus allows for a detection of glyphosate over a wide concentration range.

In embodiments of the invention, the surface is designed to be substantially planar. For example, the surface can be a moulded glass body or a moulded plastics material body, for example a slide, cover slip, silicon wafer or the like. The surface is preferably a moulded glass body.

In embodiments of the invention, the surface is designed to be transparent, semitransparent or opaque. The surface is preferably designed to be transparent at least in the wavelength range from 400 nm to 600 nm.

In embodiments of the invention, the detection takes place by means of quartz crystal microbalance (QCM), surface plasmon spectroscopy (SPR), atomic force spectroscopy or impedance spectroscopy.

In embodiments of the invention, the detection takes place by means of reflection interference contrast microscopy (RICM). The contact radii a of particles and surface as well as the particle radii R_{HES} are automatically ascertained by means of software specially developed for this purpose. According to the Johnson-Kendall-Roberts model [2], these quantities are related to the adhesion energy W_{adh} in the following context:

$${W}_{adh} = \frac{\frac{4}{3} {a}^{3} {E}_{H G S} / ( 1 - {v}^{2} )}{6 π {R}_{H G S}^{2}}$$

The ascertained adhesion energy corresponds to the binding density of the particle-bound competitors to the analyte binding partner and allows the determination of the amount of analyte in the sample.

The invention is also a surface having the analyte binding partner according to the invention, wherein the analyte binding partner is a fusion protein having a hydrophobin domain and a 5-enolpyruvylshikimate-3-phosphate synthase domain, wherein the surface is designed to be transparent at least in the wavelength range from 400 nm to 600 nm.

In embodiments of the surface according to the invention, the analyte binding partner is designed as a fusion protein. The fusion protein has, for example, different protein domains which have different functionalities.

In embodiments of the surface according to the invention, the analyte binding partner comprises a protein domain having an analyte binding site. The analyte binding site is used to bind the analyte to the analyte binding partner.

In embodiments of the surface according to the invention, the analyte binding site is designed as a binding pocket of an enzyme or an allosteric binding site for the analyte.

The analyte binding partner preferably has the active centre of the enzyme 5-enolpyruvylshikimate-3-phosphate synthase (EPSPS) as the analyte binding site. The distance between the binding pocket and the protein surface in the EPSPs is approx. 10 Å in the closed conformation.

In embodiments of the surface according to the invention, the analyte binding partner comprises a protein domain selected from hydrophobins, ECM proteins, S-layer proteins, peptide linkers, and protein tags. This allows directed immobilisation to the surface, at least one protein domain mediating the binding and a further protein domain having a further functionality. The analyte binding partner preferably has a hydrophobin domain.

In embodiments of the surface according to the invention, the analyte binding partner has a hydrophobin domain Ccg2 from Neurospora (N.) crassa. The analyte binding partner preferably has SEQ. ID. No. 5.

According to the invention, the analyte binding partner is a fusion protein having a hydrophobin domain and a 5-enolpyruvylshikimate-3-phosphate synthase domain. The hydrophobin domain allows for the immobilisation of the analyte binding partner on the surface. In embodiments of the surface according to the invention, the fusion protein has SEQ ID No. 6.

The use of a fusion protein having a hydrophobin domain advantageously results in a directed and controlled assembly of the hydrophobin or fusion protein on the surface and the EPSPS domain has no direct contact with the surface material, so that the enzymatic activity is largely retained.

In embodiments of the surface according to the invention, the analyte binding partner is immobilised on the surface in a mixture with hydrophobins, the mixture of the analyte binding partner with the hydrophobin being in the range from 1:2 to 1:10, preferably between 1:3 to 1:8, particularly preferably around 1:5. Advantageously, by using the mixing ratios of the analyte binding partner with hydrophobin, steric hindrance of the analyte binding partner, in particular the 5-enolpyruvylshikimate-3-phosphate synthase domain, is minimised by immobilisation on the surface and the responsiveness and sensitivity of the surface or the assay are modulated.

In embodiments of the surface according to the invention, a mixture of the fusion protein having a hydrophobin domain and a 5-enolpyruvylshikimate-3-phosphate synthase domain (SEQ ID No. 6) and the hydrophobin domain Ccg2 from Neurospora (N.) crassa (SEQ ID No. 5) takes place in a ratio of 1:2 to 1:10, preferably between 1:3 to 1:8, particularly preferably around 1:5, most preferably 1:5 for the immobilisation of the analyte binding partner.

The invention also relates to a functionalised particle having an immobilised competitor, wherein the competitor is immobilised on the particle via a linker, wherein the particle is designed to be deformable, wherein the competitor is selected from phosphoenolpyruvate, phosametine, substrate analogues of the enzyme 5-enolpyruvylshikimate-3-phosphate synthase or glyphosate, wherein the linker has a contour length in the range from 5 Å to 200 Å, preferably in the range from 10 Å to 50 Å and/or has a degree of polymerisation in the range from 1 to 70, preferably in the range from 3 to 20.

In embodiments of the invention, the particle has a carboxy functionalisation. The synthesis and carboxy functionalisation of the particles, in particular the hydrogel microparticles, took place according to the method described by Pussak et al. (Pussak, et al., 2012) via emulsion and radical precipitation polymerisation of polyethylene glycol diacrylamide or polyethylene glycol diacrylate with subsequent radical grafting of acrylic acid monomers, crotonic acid monomers or other alkene derivatives with functional groups such as amines for the introduction of the carboxyl, amino or other functional groups.

In embodiments, the synthesis and carboxy functionalisation of the particles takes place microfluidically by means of photoinitiated radical crosslinking of polyethylene glycol diacrylamide or polyethylene glycol diacrylate (preferred molecular weight in the range from 500 Da to 8,000 Da, particularly preferably around 4,000 Da) with subsequent photoinitiated radical grafting of acrylic acid monomers to introduce the carboxyl groups, monodisperse particles having a diameter from 10 μm to 100 μm, particularly preferably 25 μm, being produced.

According to the invention, the particle is designed to be a deformable particle. The particle preferably has a modulus of elasticity in the range from 10 kPa to 100 kPa, particularly preferably in the range from 15 kPa to 50 kPa. In this way, a high level of sensitivity is advantageously ensured. At the same time, uneven deformation of the particles is excluded.

In embodiments of the invention, the particle is a hydrogel particle.

In embodiments of the invention, the particle has a diameter of 10 μm to 100 μm, particularly preferably a diameter of around 25 μm.

According to the invention, the competitor is selected from phosphoenolpyruvate, phosametine (Huangcaoling), substrate analogues of the enzyme 5-enolpyruvylshikimate-3-phosphate synthase (EPSPS) or glyphosate. The competitor glyphosate is preferred.

According to the invention, the competitor is immobilised on the particle via a linker. The linker is attached to the functionalised particle surface.

The respective linker molecule allows suitable immobilisation of the competitor, such as glyphosate, via the carboxyl or secondary amino group, the coupling group influencing the functionality and sensitivity of the method. Furthermore, the resulting affinity of the immobilised competitor can be varied via the length or the degree of polymerisation of the linker and thus the working range of the method can be set.

In embodiments of the invention, the linker is selected from the groups of homo- and heterobifunctional linkers and comprises, for example, ethylenediamine, oligo- and polyethylene glycol bisamines, peptides such as pentaglycine and amino acids, or a bifunctional linker having other groups such as thiols or azides.

In embodiments of the invention, the linker has a contour length (L) and a degree of polymerisation (PG). According to the invention, the linker has a contour length (L) in the range from 5 Å to 200 Å, preferably in the range from 10 Å to 50 Å.

According to the invention, the linker has a degree of polymerisation in the range from 1 to 70, preferably in the range from 3 to 20.

Examples of suitable linkers are ethylenediamine: L=5.3 Å; PG=1 or pentaglycine: L=16.3 Å; PG=5 or PEG-bisamine 3000: L=191 Å; PG=68.

The resulting affinity of the immobilised competitor for the analyte binding partner can advantageously be varied via the length or the degree of polymerisation of the linker and thus the working range of the sensor can be set.

In embodiments of the invention, the linkers contain protecting groups. In embodiments of the invention, the protecting group is selected from fluorenylmethoxycarbonyl, tert-butyloxycarbonyl or tert-butyl protecting groups. In this way, it is advantageously ensured that no undesired polymerisation of the linker molecules or cross-linking of the particles occurs.

The invention is also a kit, comprising:
at least one immobilised analyte binding partner or
an analyte binding partner and a hydrophobin,
wherein the analyte binding partner is designed to interact with an analyte and is immobilised on a surface,
wherein the analyte binding partner is a fusion protein having a hydrophobin domain and a 5-enolpyruvylshikimate-3-phosphate synthase domain, and
wherein the surface is designed to be transparent at least in the wavelength range from 400 nm to 600 nm;
at least one particle, having an immobilised competitor,
wherein the competitor is immobilised on the particle via a linker, wherein the particle is designed to be deformable.

In embodiments, the analyte binding partner contained in the kit and the hydrophobin are advantageously mixed for immobilisation in a ratio of 1:2 to 1:10, preferably 1:3 to 1:8, particularly preferably in a ratio of 1:5, and immobilised on a surface. The hydrophobin stabilises the surface while the analyte binding partner is designed to interact with the analyte.

In embodiments of the kit according to the invention, the competitor is selected from phosphoenolpyruvate, phosametine (Huangcaoling), substrate analogues of the enzyme 5-enolpyruvylshikimate-3-phosphate synthase (EPSPS) or glyphosate. The competitor glyphosate is preferred.

In embodiments of the kit according to the invention, the particle is a functionalised particle, the surface of the particle having a functionalisation. The functionalisation is used to bind the competitor.

In embodiments of the kit according to the invention, the particle has a carboxy functionalisation. The synthesis and carboxy functionalisation of the particles, in particular the hydrogel microparticles, took place according to the method described by Pussak et al. (Pussak, et al., 2012) via emulsion and radical precipitation polymerisation of polyethylene glycol diacrylamide or polyethylene glycol diacrylate with subsequent radical grafting of acrylic acid monomers, crotonic acid monomers or other alkene derivatives with functional groups such as amines for the introduction of the carboxyl, amino or other functional groups.

In embodiments, the synthesis and carboxy functionalisation of the particles takes place microfluidically by means of photoinitiated radical crosslinking of polyethylene glycol diacrylamide or polyethylene glycol diacrylate (preferred molecular weight in the range from 500 Da to 8,000 Da, particularly preferably around 4,000 Da) with subsequent photoinitiated radical grafting of acrylic acid monomers to introduce the carboxyl groups, monodisperse particles having a diameter from 10 to 100 μm, particularly preferably 25 μm, being produced.

According to the invention, the competitor is immobilised on the particle via a linker. The linker is attached to the functionalised particle surface.

The respective linker molecule allows suitable immobilisation of the competitor, such as glyphosate, via the carboxyl or secondary amino group, the coupling group influencing the functionality and sensitivity of the method. Furthermore, the resulting affinity of the immobilised competitor can be varied via the length or the degree of polymerisation of the linker and thus the working range of the method can be set.

In embodiments of the kit according to the invention, the linker is selected from the groups of homo- and heterobifunctional linkers and comprises, by way of example, ethylenediamine, oligo- and polyethylene glycol bisamines, peptides such as pentaglycine and amino acids, or a bifunctional linker having other groups such as thiols or azides.

In embodiments of the kit according to the invention, the linkers contain protecting groups. In embodiments of the kit according to the invention, the protecting group is selected from fluorenylmethoxycarbonyl, tert-butyloxycarbonyl, or tert-butyl protecting groups. In this way, it is advantageously ensured that no undesired polymerisation of the linker molecules or cross-linking of the particles occurs.

In embodiments of the kit according to the invention, the linker has a contour length (L) and a degree of polymerisation (PG). In embodiments of the kit according to the invention, the linker has a contour length (L) in the range from 5 Å to 200 Å, preferably in the range from 10 Å to 50 Å.

In embodiments of the kit according to the invention, the linker has a degree of polymerisation in the range from 1 to 70, preferably in the range from 3 to 20.

Examples of suitable linkers are ethylenediamine: L=5.3 Å; PG=1 or pentaglycine: L=16.3 Å; PG=5 or PEG-bisamine 3000: L=191 Å; PG=68.

In embodiments of the kit according to the invention, the particle is a hydrogel particle.

In embodiments of the kit according to the invention, the particle size is a diameter in the range from 10 μm to 100 μm, particularly preferably around 25 μm.

In embodiments of the kit according to the invention, the particle has a modulus of elasticity in the range from 5 kPa to 100 kPa, particularly preferably in the range from 15 kPa to 50 kPa. In this way, a high level of sensitivity is advantageously ensured. At the same time, uneven deformation of the particles is excluded.

In embodiments of the kit according to the invention, the surface is designed to be substantially planar. For example, the surface can be a moulded glass body, for example a slide, cover slip, silicon wafer or the like.

In embodiments of the kit according to the invention, the surface is designed to be transparent, semitransparent or opaque.

According to the invention, the analyte binding partner is designed as a fusion protein. The fusion protein has, for example, different protein domains which have different functionalities.

According to the invention, the analyte binding partner comprises a protein domain having an analyte binding site. The analyte binding site is used to bind the analyte to the analyte binding partner.

In embodiments of the kit according to the invention, the analyte binding site is designed as a binding pocket of an enzyme or an allosteric binding site for the analyte.

The analyte binding partner preferably has the active centre of the enzyme 5-enolpyruvylshikimate-3-phosphate synthase (EPSPS) as the analyte binding site. The distance between the binding pocket and the protein surface in the EPSPs is approx. 10 Å in the closed conformation.

In embodiments of the kit according to the invention, the analyte binding partner comprises a protein domain selected from hydrophobins, ECM proteins, S-layer proteins, peptide linkers, and protein tags. This allows directed immobilisation to the surface, at least one protein domain mediating the binding and a further protein domain having a further functionality. The analyte binding partner preferably has a hydrophobin domain.

In embodiments of the kit according to the invention, the analyte binding partner has a hydrophobin domain Ccg2 from Neurospora (N.) crassa. The analyte binding partner preferably has SEQ. ID. No. 5.

According to the invention, the analyte binding partner is a fusion protein having a hydrophobin domain and a 5-enolpyruvylshikimate-3-phosphate synthase domain. The hydrophobin domain allows for the immobilisation of the analyte binding partner on the surface. In embodiments of the kit according to the invention, the fusion protein has SEQ ID No. 6.

In embodiments of the kit according to the invention, the analyte binding partner is immobilised on the surface in a mixture with hydrophobins, the mixture of the analyte binding partner with the hydrophobin being in the range from 1:2 to 1:10, preferably between 1:3 to 1:8, particularly preferably around 1:5.

In embodiments of the kit according to the invention, a mixture of the fusion protein having a hydrophobin domain and a 5-enolpyruvylshikimate-3-phosphate synthase domain (SEQ ID No. 6) and the hydrophobin domain Ccg2 from Neurospora (N.) crassa (SEQ ID No. 5) takes place in a ratio of 1:2 to 1:10, preferably between 1:3 to 1:8, particularly preferably around 1:5, most preferably 1:5 for the immobilisation of the analyte binding partner.

The invention also relates to the use of the method according to the invention, the surface according to the invention, the particle according to the invention and the kit according to the invention for the detection of analytes in samples such as water, food, soil, drinking and wastewater samples, preferably in aqueous solutions.

In order to implement the invention, it is also expedient to combine the above-described embodiments and individual features of the claims.

### EMBODIMENTS

Further features and advantages of the present invention emerge from the following schematic drawings and embodiments, on the basis of which the invention is to be explained in more detail by way of example without restricting the invention thereto.

In the drawings:

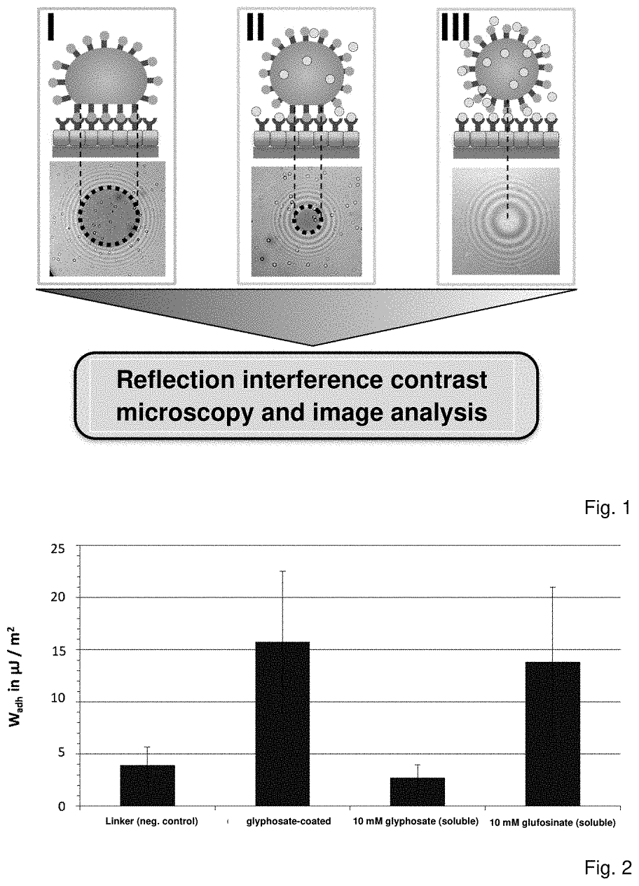

FIG. 1: is the schematic representation of the method according to the invention based on the competitive binding of particle-bound and soluble glyphosate to the protein-functionalised surface;

FIG. 2: shows the dependence of the adhesion energy between particle and surface on the concentration of glyphosate in solution and soluble glufosinate as a negative control;

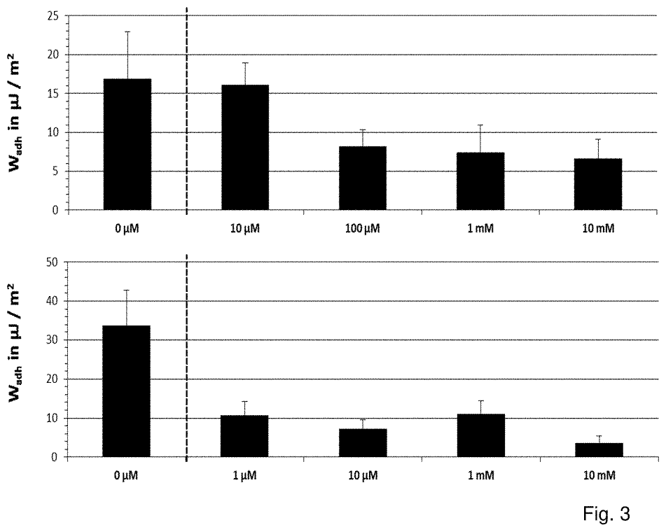

FIG. 3: shows the dependence of the limit of detection and the working range of the method on the coating of the particles;

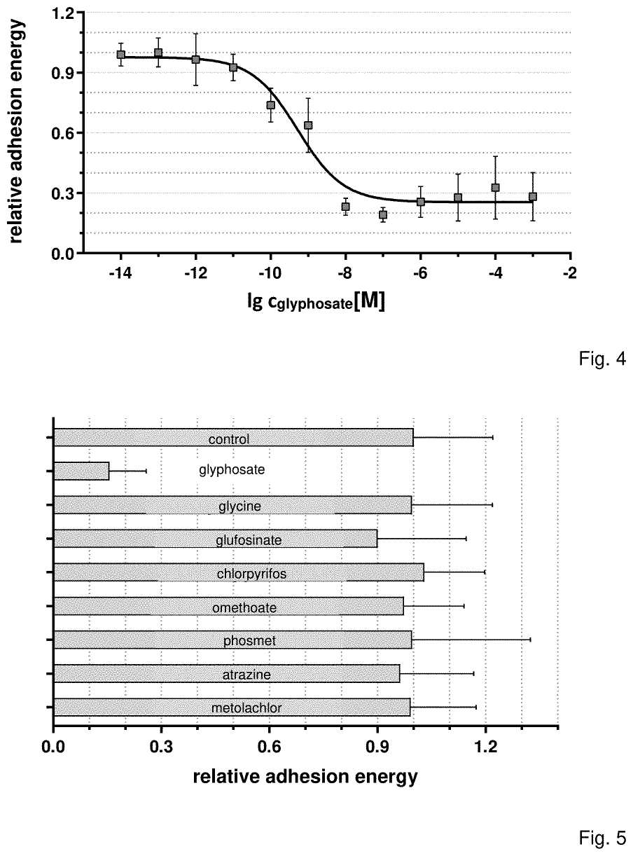

FIG. 4: shows the relative adhesion energy of the particles coated with glyphosate bound via a pentaglycine linker depending on the glyphosate concentration (soluble);

FIG. 5: shows the comparison of the relative adhesion energies of particles coated with pentaglycine glyphosate on glyphosate, other pesticides as well as glycine as a structural element of glyphosate, tested in each case at a concentration of 1 mM, except for atrazine (153 μM), chlorpyrifos (4 μM) and phosmet (79 μM).

The principle of the detection method is shown in FIG. 1. The immobilised enzymes on the surface interact attractively with the particle-bound competitor, resulting in a characteristic contact area between the surface and the particle. The dissolved analyte competes with the competitor for the binding sites on the surface. This reduces the contact area depending on the concentration of the analyte. Above a specific concentration, the particles do not adhere to the surface. The determination of the contact and particle radius to ascertain the adhesion energy takes place by means of reflection interference contrast microscopy.

For the detection of the specificity of the method, RCA-cleaned glass surfaces were first coated in FIG. 2 with a suitable mixture of the hydrophobin Ccg2 and the fusion protein thereof Ccg2_GS_EcEPSPS (SEQ ID No. 6). The surfaces were then incubated with either 10 mM glyphosate or glufosinate solutions and then with linker-functionalised particles with or without glyphosate coating. The adhesion energies resulting from the respective conditions were shown on the basis of a representative data set.

In FIG. 3, RCA-cleaned glass surfaces were first coated with a suitable mixture of the hydrophobin Ccg2 and the fusion protein thereof Ccg2_GS_EcEPSPS (SEQ ID No. 6). The surfaces were then incubated with glyphosate solutions of different concentrations and then with glyphosate-coated particles with ethylenediamine or pentaglycine linkers. The adhesion energies resulting from the respective conditions were shown on the basis of a representative data set.

### Embodiment 1: Quantification of Glyphosate by Means of Competitive Binding

To create a functionalised surface, fusion proteins from the hydrophobin Ccg2 (SEQ ID No. 5) from Neurospora (N.) crassa and the 5-enolpyruvylshikimate-3-phosphate synthase from Escherichia (E.) coli (EcEPSPS) (SEQ ID No. 4) were required. For this purpose, the coding regions of the respective genes, linked via the sequence for a flexible glycine-serine linker, were transferred to the expression vector pET28b (Novagen, Germany). In this case, the sequence of the hydrophobin (SEQ ID No. 2) is on the 5′ side and the sequence of the EcEPSPS (SEQ ID No. 1) is on the 3′ side of the linker sequence. In addition, on the 5′ side of the sequence for the fusion protein, there is the sequence for a (His)₆ tag, which is required for the detection and purification of the fusion protein. Furthermore, the hydrophobin without EcEPSPS was required for the surface. The gene sequence was accordingly transferred into the vector pET28b without the linker and the EcEPSPS sequence. The vectors modified in this way were, after complete sequencing of the sequences introduced, transferred into the E. coli expression strain SHuffle T7 Express lysY (New England Biolabs, USA). This expression strain is advantageous for the expression of the hydrophobins, since it also codes for a disulphide bridge isomerase, which promotes the correct formation of the disulphide bridges of the hydrophobins. These play a substantial role in the correct folding of the protein.

Protein expression was started by adding 1 mM isopropyl-β-D-thiogalactoside (IPTG) to E. coli cells which were in the exponential growth phase. After induction, the cells were incubated for a further 4 hours at 30° C. and 180 revolutions per minute. The cells were then pelleted and washed so that they can then be used for protein purification. The purification method used depends on the solubility of the proteins. The soluble fusion proteins (SEQ ID No. 6) were purified by means of nickel affinity chromatography under native conditions according to the manufacturer's instructions, while the insoluble hydrophobins were purified by means of denaturing nickel affinity chromatography according to the manufacturer's instructions. The hydrophobins were concentrated by ultrafiltration before dialysis; this was not necessary for the fusion proteins. After purification, the hydrophobins were dialysed against a redox refolding buffer (10 mM glutathione reduced, 1 mM glutathione oxidised; pH 5.4) and the fusion proteins were dialysed against the Monsanto dialysis buffer (10 mM MOPS, 0.5 mM EDTA, 5% (v/v) 99.9% glycerol, 1 mM DTT, pH 7), then stored in the refrigerator and used for the functionalisation of glass surfaces.

The functionalisation of glass surfaces took place by slowly pipetting on the protein solution with subsequent incubation for 30 minutes at room temperature. The surfaces were then washed thoroughly with distilled water and dried for 30 minutes at RT. Before the functionalisation, the glass surfaces were cleaned with the aid of an RCA solution (50 ml of 25% strength aqueous NH₃ solution, 50 ml of 35% H₂O₂, 250 ml of deionised water).

The two protein variants were needed to find an optimal ratio between fusion protein (can bind glyphosate) and hydrophobin (to stabilise the surface) for the method. For this purpose, the proteins were mixed in different proportions and the functionality of the EcEPSPS on the surface was determined by means of a detection of inorganic phosphate. Inorganic phosphate is one of the reaction products in the reaction of EPSPS with its substrates phosphoenolpyruvate (PEP) and shikimate-3-phosphate (S3P) and can therefore be used for the detection of enzyme activity. The ratio of Ccg2 (SEQ ID No. 5) to Ccg2_GS_EcEPSPS (SEQ ID No. 6) affects the sensitivity and signal strength of the assay. The more fusion protein there is on the surface, the more glyphosate is needed to occupy the binding sites and thus measurably inhibit the activity of the proteins on the surface. Accordingly, a surface with a high concentration of fusion protein is less sensitive than one with little fusion protein. However, too low a concentration of the fusion protein results in a low signal-to-noise ratio. For this reason, various occupancy ratios were tested, resulting in a ratio of 1 μM of Ccg2_GS_EcEPSPS (SEQ ID NO. 6) to 5 μM of Ccg2 (SEQ ID No. 5) as well suited for the application.

The synthesis and carboxy functionalisation of the hydrogel microparticles took place according to the method described by Pussak et al. via emulsion and radical precipitation polymerisation of polyethylene glycol diacrylamide with subsequent radical grafting of acrylic acid monomers for the introduction of the carboxyl groups [1]. For the methods explained below, microparticles with moduli of elasticity of 15 kPa and a mean radius of 20 μm were used.

In order to allow for an interaction between the surface and the hydrogel particles or hydrogel probes (HGS) that can be modulated by the presence of the analyte, the microparticles were coated with glyphosate. For this purpose, starting from the carboxyl-functionalised HGS, various linkers were coupled to the particles. The respective linker molecule allows suitable immobilisation of the glyphosate via the carboxyl or secondary amino group, the coupling group influencing the functionality and sensitivity of the sensor. Furthermore, the resulting affinity of the immobilised competitor for the enzyme (EPSPS) can be varied via the length or the degree of polymerisation of the linker and thus the working range of the sensor can be set.

The respective coupling steps were carried out using active ester chemistry. Ethylenediamine served as a short linker variant, which was coupled to the microparticles by means of benzotriazole-1-yl-oxy-tris-pyrrolidino-phosphonium hexafluorophosphate (PyBOP) and 1-hydroxybenzotriazole (HOBt). For this purpose, the particles were suspended and water was replaced by dimethylformamide (DMF) in a plurality of washing steps. The particles were left in 2 ml of DMF. Subsequently, 146 mg (280 μmol) of PyBOP, 18 mg (140 μmol) of HOBt and 39 μl (280 μmol) of triethylamine were added for the activation of the carboxyl groups and the suspension was shaken at room temperature for 1 h. After adding 20 μl (300 μmol) of ethylenediamine and reacting for three hours, the particles were centrifuged at 1844×g for 10 min and in each case washed 3 times with DMF, a 1:1 mixture of DMF and water as well as pure water. For further coating, 4 mg (24 μmol) of glyphosate were dissolved in 2 ml 100 mM of Hepes buffer (pH=7.0) in an ultrasonic bath and 46 mg (240 μmol) of 1-ethyl-3-(3-dimethylaminopropyl)carbodiimide-hydrochloride (EDC) and 52 mg (240 μmol) of N-hydroxy-sulfosuccinimide sodium salt (s-NHS) were added and the carboxyl groups were activated for 15 min. The coupling of the pesticide to the amine-functionalised particles took place by combining suspension and solution and reacting over a period of 1 hour. Finally, the particles were washed 3 times with a 100 mM Hepes buffer solution (pH=7.0).

Alternatively, the particles were functionalised with a pentaglycine linker. For this purpose, the particles were washed 3 times with 2 ml of 100 mM MES buffer (pH=5.3) and the supernatant was discarded after centrifugation (10 min, 1844×g). 23 mg (120 μmol) of EDC and 26 mg (120 μmol) of s-NHS were dissolved in 1 ml of 100 mM MES buffer (pH=5.3) and the microparticles were then suspended in the solution. After activation of the carboxyl groups for one hour while shaking, 10 μl (143 μmol) of mercaptoethanol were added to inactivate the excess EDC and the suspension was left at room temperature for a further 15 min. After centrifugation (10 min, 1844×g), the supernatant was discarded and 0.2 mg (660 nmol) of the peptide dissolved in 1 ml of Hepes buffer solution (100 mM) was added, the coupling of the linker taking place overnight. After a further 3 washing steps (100 mM of Hepes buffer), the carboxyl groups were converted into the s-NHS ester according to the procedure described above, excess EDC was inactivated by means of mercaptoethanol, the suspension was centrifuged and the supernatant was discarded. After adding a 1 mg/ml of 100 mM Hepes-buffered glyphosate solution (6 μmol), the reaction mixture was left at room temperature overnight with shaking. Finally, the particles were washed 3 times with a 100 mM Hepes buffer solution (pH=7.0).

After the surface, for example a glass surface, and the particles had been coated, they could be used for reflection interference contrast microscopy (RICM). For this purpose, the surfaces according to the invention were glued to a 16-well carrier (CS16-CultureWell™, Grace Biolabs) with a self-adhesive underside and a volume of 400 μl/well.

200 μl/well of analyte solution (100 mM HEPES buffer pH=7) were then added. After incubating the surfaces for 30 minutes, 10 μl/well of the hydrogel particles functionalised with glyphosate were added and the surfaces were microscoped after the hydrogel particles had sedimented.

The recording of the radial intensity profiles of the HGS on the functionalised surface took place in the reflection interference contrast method by means of an inverted microscope system (Olympus IX 73) with a 60× immersion objective (Olympus UPIanSAPO 60× Oil Microscope Objective). From the recorded profiles, the contact radii a of particles and surface as well as the particle radii R_{HES} could be automatically ascertained subsequently by means of software specially developed for this purpose. According to the Johnson-Kendall-Roberts model [2], these quantities are related to the adhesion energy W_{adh} in the following context:

$${W}_{adh} = \frac{\frac{4}{3} {a}^{3} {E}_{H G S} / ( 1 - {v}^{2} )}{6 π {R}_{H G S}^{2}}$$

With a modulus of elasticity E_{HGS} of the particles of 15 kPa and a Poisson's number v of 0.5, the corresponding adhesion energy of the system can thus be determined with knowledge of the particle and contact radius.

The results of the measurements are shown by way of example in FIG. 2. The pentaglycine-functionalised particles (negative control) show only weak, unspecific interactions with the surface, whereas glyphosate-coated HGS adhere strongly. In the presence of high concentrations of the analyte, the value is in the range of the negative control. This is due to the competition for binding sites of the EcEPSPS on the surface between free glyphosate in the analyte solution and glyphosate bound to the HGS. Furthermore, the negligible influence of structurally similar compounds such as glufosinate on the adhesion energy illustrates the selectivity of the method compared to glyphosate.

FIG. 3 shows an example of the resulting adhesion energies of glyphosate-coated particles of the linker variants ethylenediamine and pentaglycine at different concentrations of soluble glyphosate. With increasing glyphosate concentrations in the sample, the adhesion energy of the glyphosate-coated HGS on the functionalised chip surface decreases. The more glyphosate there is in the analyte solution, the stronger the competition with bound glyphosate. If many binding sites are occupied with free glyphosate, the HGS can no longer adhere firmly to the surface, the contact area becomes smaller and the adhesion energy decreases accordingly. The limit of detection can be varied, among other things, via the linker. In the example shown, the limit of detection is 10 μM for ethylenediamine-glyphosate-coated HGS, for pentaglycine-glyphosate-coated HGS the limit of detection is below 1 μM, whereby the sensor system offers further options for setting the working range. The results show that specific detection and precise quantification of glyphosate are possible with the aid of the invention.

### Embodiment 2: Determination of the Sensitivity and Specificity of the Quantification of Glyphosate

The concentration dependence of the glyphosate binding is shown in FIG. 4. For the determination of the sensitivity, RCA-cleaned glass surfaces were first coated with a suitable mixture of the hydrophobin Ccg2 and the fusion protein thereof Ccg2_GS_EcEPSPS (SEQ ID No. 6). The surfaces were then incubated with glyphosate solutions of different concentrations and then with glyphosate-coated particles with pentaglycine linkers. The examined concentration range covers a range from 10⁻¹¹ M to 10⁻⁸ M. This sensitivity range reaches the threshold value of 0.1 μg/ml for pesticide contamination in German tap water.

The specificity was examined by testing structurally related compounds and frequently used pesticides in the assay according to the invention (FIG. 5). None of the tested compounds showed a relative adhesion energy comparable to that of glyphosate, whereby a specific detection of glyphosate was also proven in the presence of other pesticides and in the presence of compounds with structural elements from glyphosate.

### CITED NON-PATENT LITERATURE

[1] D. Pussak, M. Behra, S. Schmidt and L. Hartmann, Soft Matter, 2012, 8, 1664
[2] K. L. Johnson, K. Kendall, A. D. Roberts, Proc. R. Soc. Lond. Ser. A—Math. Phys. Sci., 1971, 324, 301
[3] M. R. Marzabadi, K. J. Gruys, P. D. Pansegrau, M. C. Walker, H. K. Yuen, J. A. Sikorski, Biochemistry, 1996, 35, 13, 4199
[4] M. A. Priestman, M. L. Healy, A. Becker, D. G. Alberg, P. A. Bartlett, G. H. Lushington, E. Schönbrunn, Biochemistry, 2005, 44, 9, 3241

1. A method for the detection of analytes comprising the steps of:
providing a surface with an immobilised analyte binding partner,
contacting the analyte binding partner with a sample containing the analyte, wherein the analyte interacts with the analyte binding partner,
contacting the analyte binding partner with a competitor, wherein the competitor is immobilised on a particle and interacts with the analyte binding partner,
detecting the competitors bound to the analyte binding partner,
wherein the analyte is glyphosate,
wherein the analyte binding partner has the active centre of the enzyme 5-enolpyruvylshikimate-3-phosphate synthase (EPSPS) as the analyte binding site, and
wherein the particle is designed to be deformable.

2. The method according to claim 1, wherein the particle is a hydrogel particle.

3. The method according to claim 1, wherein the particle has a modulus of elasticity of 5 kPa to 100 kPa.

4. The method according to claim 1, wherein the competitor is selected from phosphoenolpyruvate, phosametine, substrate analogues of the enzyme 5-enolpyruvylshikimate-3-phosphate synthase or glyphosate.

5. The method according to claim 1, wherein the analyte binding partner is a fusion protein having a hydrophobin domain and a 5-enolpyruvylshikimate-3-phosphate synthase domain.

6. The method according to claim 1, wherein the fusion protein has SEQ ID No. 6.

7. The method according to claim 1, wherein the analyte binding partner is immobilised on the surface in a mixture with hydrophobins, the mixture of the analyte binding partner with the hydrophobin being in the range from 1:2 to 1:10.

8. The method according to claim 1, wherein the detection takes place by means of reflection interference contrast microscopy.

9. A surface having an analyte binding partner, wherein the analyte binding partner is a fusion protein having a hydrophobin domain and a 5-enolpyruvylshikimate-3-phosphate synthase domain, wherein the surface is designed to be transparent at least in the wavelength range from 400 nm to 600 nm.

10. The surface according to claim 9, wherein the analyte binding partner is immobilised on the surface in a mixture with hydrophobins, the mixture of the analyte binding partner with the hydrophobin being in the range from 1:2 to 1:10.

11. The surface according to claim 9, wherein the analyte binding partner is a fusion protein which has SEQ ID No. 6.

12. A particle having an immobilised competitor, wherein the competitor is immobilised on the particle via a linker, wherein the particle is designed to be deformable, wherein the competitor is selected from phosphoenolpyruvate, phosametine, substrate analogues of the enzyme 5-enolpyruvylshikimate-3-phosphate synthase or glyphosate,
wherein the linker has a contour length of 5-200 Å and/or a degree of polymerisation of 1-70.

13. A kit, comprising:
at least one immobilised analyte binding partner or one analyte binding partner and a hydrophobin, wherein the analyte binding partner is designed to interact with an analyte and is immobilised on a surface,
wherein the analyte binding partner is a fusion protein having a hydrophobin domain and a 5-enolpyruvylshikimate-3-phosphate synthase domain, and
wherein the surface is designed to be transparent at least in the wavelength range from 400 nm to 600 nm; and
at least one particle, having an immobilised competitor,
wherein the competitor is immobilised on the particle via a linker,
wherein the particle is designed to be deformable.

14. The kit according to claim 13, wherein the fusion protein has SEQ ID No. 6.

15. The kit according to claim 13, wherein the analyte binding partner is immobilised on the surface in a mixture with hydrophobins, the mixture of the analyte binding partner with the hydrophobin being in the range from 1:2 to 1:10, preferably between 1:3 to 1:8, particularly preferably around 1:5.

16. The method according to claim 1, wherein the analyte binding partner is immobilised on the surface in a mixture with hydrophobins, the mixture of the analyte binding partner with the hydrophobin being in the range from 1:3 to 1:8.

17. The method according to claim 1, wherein the analyte binding partner is immobilised on the surface in a mixture with hydrophobins, the mixture of the analyte binding partner with the hydrophobin being around 1:5.

18. The surface according to claim 9, wherein the analyte binding partner is immobilised on the surface in a mixture with hydrophobins, the mixture of the analyte binding partner with the hydrophobin being in the range from 1:3 to 1:8.

19. The surface according to claim 9, wherein the analyte binding partner is immobilised on the surface in a mixture with hydrophobins, the mixture of the analyte binding partner with the hydrophobin being around 1:5.

20. The particle according to claim 12, wherein the linker has a contour length of 10-50 Å and/or a degree of polymerisation of 3-20.
